(19) United States
(12) Patent Application Publication
(10) Pub. No.: US 2023/0227816 A1
(43) Pub. Date: Jul. 20, 2023

(54) METHODS AND COMPOSITIONS FOR SELECTIVE PCR AND CLONING OF ANTIBODY SEQUENCES

(71) Applicants: CHAN ZUCKERBERG BIOHUB, INC., San Francisco, CA (US); THE BOARD OF TRUSTEES OF THE LELAND STANFORD JUNIOR UNIVERSITY, Stanford, CA (US)

(72) Inventors: Richard Felix Horns, Stanford, CA (US); Stephen R. Quake, San Francisco, CA (US)

(21) Appl. No.: 18/009,549

(22) PCT Filed: Jun. 15, 2021
(86) PCT No.: PCT/US21/37414

§ 371 Date: Dec. 9, 2022

Related U.S. Application Data
(60) Provisional application No. 63/039,113, filed on Jun. 15, 2020.

(51) Int. Cl. C12N 15/10; C12N 15/63; C12Q 1/686; C07K 16/00

(52) U.S. Cl. CPC C12N 15/1096; C12N 15/63; C12Q 1/686; C07K 16/005; C07K 2317/10; C12N 2330/50

(57) ABSTRACT

The present disclosure provides materials and methods for cloning antibodies from single cells in pooled sequence libraries by selective PCR. The compositions and methods relate to isolating, cloning, and/or expressing one or more antibody sequences from a single cell from a pool of cells.

### CROSS-REFERENCE TO RELATED APPLICATIONS

The present application claims priority to U.S. Provisional Patent Application No. 63/039,113 filed Jun. 15, 2020 the entirety of which is incorporated by reference herein.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “55621_Seqlisting.txt”, which was created on Jun. 15, 2021 and is 22,274 bytes in size. The subject matter of the Sequence Listing is incorporated herein in its entirety by reference.

### FIELD

The present disclosure relates generally to methods for cloning antibodies from single cells in pooled sequence libraries by selective PCR.

### BACKGROUND

Antibodies enable immune recognition by binding to target molecules called antigens. Characterization of antibody binding properties, such as specificity and affinity, is essential for understanding the recognition capability of the immune system and discovering antibodies for research and therapeutics. Currently, sequence information alone is not sufficient to predict antibody specificity and affinity. Thus, characterization of antibody binding requires recombinant cloning and expression of purified protein for use in functional assays.

Single-cell approaches enable high-throughput determination of native antibody sequences, but remain inadequate for functional characterization at similar scale. Droplet- and microwell-based single-cell sequencing techniques can identify >10,000 natively paired antibody heavy- and light-chain gene sequences per experiment (DeKosky, B. J., et al. (2013), Nat Biotechnol 31, 166-169; Goldstein et al., 2019, Commun Biol 2, 1-10; Horns et al., 2020, Cell Reports 30, 905-913.e6; and McDaniel et al., 2016, Nat Protoc 11, 429-442). However, current methods yield complementary DNA (cDNA) pooled from thousands of cells, rendering isolation of antibody cDNA from individual cells difficult. Based on sequence information, antibody DNA can be produced by gene synthesis (Croote et al., 2018, Science 362, 1306-1309; Horns et al., 2020), but this approach is more costly and time-consuming than cDNA cloning. Single B cell sorting and reverse transcription-polymerase chain reaction (RT-PCR) directly yields antibody cDNA suitable for cloning and expression (Tiller et al., 2008, J Immunol Methods 329, 112-124), but this approach lacks sufficient throughput to survey antibody sequence diversity at the scale of the immune repertoire. Thus, existing methods do not permit simultaneous high-throughput determination of antibody sequences and the cloning and expression of individual antibodies for functional characterization.

### SUMMARY OF THE INVENTION

The present disclosure provides, in various aspects, methods and materials for cloning antibodies from single cells in pooled sequence libraries by selective PCR.

In one embodiment of the present disclosure, a method of cloning an antibody from a single cell is provided comprising: a. isolating a single cell from a population of cells; b. separately amplifying a heavy chain complementary DNA (cDNA) and a light chain cDNA from said single cell, wherein each amplification comprise two polymerase chain reactions (PCR), wherein the first PCR reaction comprises an outer forward primer capable of specifically hybridizing to a barcode, and an outer reverse primer capable of specifically hybridizing to the antibody constant region, and wherein the second PCR reaction comprises an inner forward primer capable of specifically hybridizing to the 5′ end of the variable region of the antibody and an inner reverse primer capable of specifically hybridizing to the 3′ end of the variable region of the antibody; and c. inserting the amplified heavy chain cDNA and the amplified light chain cDNA of step (b) into separate vectors, thereby cloning an antibody from a single cell.

In another embodiment, the two PCR reactions comprise a nested PCR process. In another embodiment, the inner reverse primer hybridizes to a region of the 3′ end of the variable region comprising CDR3.

In another embodiment of the present disclosure, the single cell is isolated from a pooled library of cells. In another embodiment, the pooled library of cells comprise B cells. In still another embodiment, each B cell comprise a unique barcode adjacent or near the 5′ end of an antibody heavy chain and an antibody light chain cDNA. In yet another embodiment, the barcode is approximately 5-50 nucleotides in length. In various other embodiments, the barcode is 5, 6, 7, 8, 9, 10, 11, 12, 13, 14, 15, 16, 17, 18, 19, 20, 21, 22, 23, 24, 25, 26, 27, 28, 29, 30 or more nucleotides in length.

In other embodiments, an aforementioned method is provided wherein the single cell is isolated by capturing the single cell in a droplet of aqueous solution using a microfluidic device. In one embodiment the solution is oil.

The present disclosure also provides in various embodiments, an aforementioned method wherein the amplified cDNA comprises a full-length variable region cDNA.

The present disclosure also provides in various embodiments, an aforementioned method wherein the vector is an expression vector. In another embodiment, introducing the vector into a cell and expressing a full-length antibody comprising a variable region and a constant region is provided. In yet another embodiment, the method further comprises purifying the antibody.

The present disclosure also provides an aforementioned method wherein the single cell is isolated from a sample from a subject. In various embodiments, the sample is a peripheral blood sample, and wherein the single cell is isolated by capturing the single cell in a droplet of aqueous solution using a microfluidic device. In still other embodiments, the sample is taken from a human subject suffering from a disease or disorder or following an infection or exposure to an antigen.

Various other methods are also contemplated by the present disclosure. For example, in one embodiments, a method of preparing an expression vector comprising an antibody sequence from a single cell is provided comprising: a. isolating a single cell from a population of cells; b. separately amplifying a heavy chain complementary DNA (cDNA) and a light chain cDNA from said single cell, wherein each amplification comprise two polymerase chase reactions (PCR), wherein the first PCR reaction comprises an outer forward primer capable of specifically hybridizing to a barcode, and an outer reverse primer capable of specifically hybridizing to the antibody constant region, and wherein the second PCR reaction comprises an inner forward primer capable of specifically hybridizing to the 5′ end of the variable region of the antibody and an inner reverse primer capable of specifically hybridizing to the 3′ end of the variable region of the antibody; and c. inserting the amplified heavy chain cDNA and the amplified light chain cDNA of step (b) into separate vectors.

In another embodiment, a method of preparing an antibody from a single cell is provided comprising: a. isolating a single cell from a population of cells; b. separately amplifying a heavy chain complementary DNA (cDNA) and a light chain cDNA from said single cell, wherein each amplification comprise two polymerase chase reactions (PCR), wherein the first PCR reaction comprises an outer forward primer capable of specifically hybridizing to a barcode, and an outer reverse primer capable of specifically hybridizing to the antibody constant region, and wherein the second PCR reaction comprises an inner forward primer capable of specifically hybridizing to the 5′ end of the variable region of the antibody and an inner reverse primer capable of specifically hybridizing to the 3′ end of the variable region of the antibody; c. inserting the amplified heavy chain cDNA and the amplified light chain cDNA of step (b) into separate vectors; and d. expressing the heavy chain cDNA and light chain cDNA from the vectors in step (c) in a host cell under conditions that allow the production of an antibody.

### BRIEF DESCRIPTION OF THE DRAWINGS

FIG. 1 shows a schematic of workflow for Selective PCR for Antibody Retrieval (SPAR). FIG. 1(A) Antibody heavy- and light-chain cDNA (IGH and IGKL, respectively) from individual cells within a pooled library are distinguished by unique sequence barcodes (SB). For example, the heavy- and light-chain cDNA from cell 1 (IGH1 and IGKL1) are marked by SB1 and SB1′. FIG. 1 (B) Molecule-specific primers are designed to target the sequence barcode. FIG. 1(C-E) Selective amplification of target molecules is performed by two-step nested PCR. Overview of nested PCR design for heavy (top) and light (bottom) chains in shown in (C) and details are shown in (D) and (E). In the first PCR step (PCR1), primers target the unique sequence barcode (SB) and constant region, labeled C. In the second PCR step (PCR2), primers target the 5′ and 3′ ends of the antibody variable region, labeled VDJ. FIG. 1(F) PCR products are cloned into linearized expression vectors in one step by Gibson assembly. FIG. 1(G) Architecture of antibody heavy- or light-chain cDNA after library preparation using 10×Genomics Chromium 5′ V(D)J platform. Sequence barcode consists of 16 bp cell barcode (CBC) and 10 bp unique molecular identifier (UMI). PCR1 forward primer targets this 26 bp sequence. (PCR, polymerase chain reaction; cDNA, complementary DNA; IGH, immunoglobulin heavy chain; IGKL, immunoglobulin kappa or lambda chain; SB, sequence barcode; CBC, cell barcode; UMI, unique molecular identifier; V, variable gene; D, diversity gene; J, joining gene; C, constant gene)

FIG. 2 shows the computational design and characteristics of SPAR primers. FIG. 2(A) Success rates of SPAR primer design for heavy chain, light chain, and both chains for all cells in test dataset (n=94,259 cells). FIG. 2(B) Number of cDNA molecules used to assemble each heavy- and light-chain contig (n=94,259 heavy-chain contigs; n=94,259 light-chain contigs). Each molecule carries a distinct unique molecular identifier (UMI). FIG. 2(C) Sequence similarity between PCR1 forward primers for all retrievable heavy-chain genes within one pooled library (n=5,647 primers), as reflected in the distribution of edit distances to the nearest neighbor. Edit distance is defined as the number of insertions, deletions, or substitutions required to change one sequence to the other, also known as Levenshtein distance. FIG. 2(D) Predicted melting temperature of forward and reverse primers for all retrievable antibodies (n=76,781). FIG. 2(E) Difference between predicted melting temperatures of forward and reverse primers in each pair for all retrievable antibodies (n=76,781). Black dot indicates median. FIG. 2(F) Positions of forward and reverse primers for all retrievable antibodies (n=76,781). Reverse primers are indicated by lighter shading, as in (D).

FIG. 3 shows the retrieval of antibodies from single cells using SPAR. FIG. 3(A) Molecular features of antibodies targeted for retrieval originating from 8 single cells chosen at random from pooled sequence libraries. Heavy- and light-chain gene usage and CDR3 length are shown. HV, heavy-chain variable; HJ, heavy-chain joining; HCDR3, heavy-chain CDR3; LV, light-chain variable; LJ, light-chain joining; LCDR3, light-chain CDR3; AA, amino acids. FIGS. 3(B and C) Agarose gel electrophoresis of SPAR PCR2 products (2% agarose) for heavy- (B) and light-chain genes (C). Expected size of each product is indicated by label at bottom. FIG. 3(D) Sequence verification of SPAR PCR2 products using Sanger sequencing for three cells. Heavy- and light-chain amino acid sequences of SPAR PCR2 products were aligned to the reference sequences obtained by single-cell sequencing. Residues matching the reference are shown in green. FIG. 3(E) Summary of SPAR products for three cells. Colonies were picked after transformation of Gibson Assembly products into E. coli, then constructs were Sanger sequenced. For each chain, the fraction of colonies with perfect (100%) identity to reference is shown. One colony with maximal identity to reference was chosen, and the amino acid (AA) and DNA identity of that colony to the reference is shown.

### DETAILED DESCRIPTION

The present disclosure addresses the aforementioned need in the art and provides methods for cloning antibodies from single cells in pooled sequence libraries by selective PCR.

Antibodies function by binding to antigens. Antibodies must be cloned and expressed to determine their binding characteristics, but current methods for high-throughput antibody sequencing yield antibody DNA pooled from many cells and do not readily permit cloning of antibodies from single B cells. The present disclosure provides a strategy for retrieving and cloning antibody DNA from single cells within a pooled library of cells. As described herein, selective PCR for antibody retrieval (SPAR), takes advantage of the unique sequence barcodes attached to individual cDNA molecules during sample preparation to enable specific amplification by PCR of antibody heavy- and light-chain cDNA originating from a single cell. Computational analysis provided herein shows that most human antibodies can be retrieved using SPAR. Experimental demonstration herein shows retrieval of full-length antibody variable region cDNA from three cells within pools of ˜6000 cells. SPAR therefore enables rapid low-cost cloning and expression of native human antibodies from pooled single-cell sequence libraries for functional characterization.

The present disclosure provides compositions and methods for isolating, cloning, and/or expressing one or more antibody sequences or one or more antibody domains from a single cell from a pool of cells. In some embodiments of the disclosure, the sequences of antibodies or antibody domains or fragments is obtained using conventional means from many cells (e.g., a pool of cells; an antibody of interest (e.g., from one cell) is identified (e.g., informatically); primers are designed (e.g., informatically) to amplify the antibody of interest; PCR (e.g., nested PCR) is used to amplify the antibody of interest; and the amplified product is then cloned into an expression vector for production and purification.

As described herein, the antibody of interest can be chosen based on a various features including, but not limited to, the clonal structure of the antibody repertoire (e.g. choose a cell from a large, expanded clone); the dynamics of the clones (e.g. choose a cell from a clone that expanded after vaccination); the genetic composition of the antibody (e.g. uses antibody V and J genes that have been associated with, for example, HIV binding); and one or more cellular features (e.g. choose an activated memory B cell). Thus, target antibodies can be chosen based on sequence or clonal characteristics, or single-cell phenotypes, such as transcriptome profile.

As used herein, “antibodies” include but are not limited to polyclonal antibodies, monoclonal antibodies (mAbs), humanized or chimeric antibodies, single chain antibodies, Fab fragments, F(ab′)2 fragments, fragments produced by a Fab expression library, and epitope binding fragments of any of the above.

“Nested PCR” is a modification of PCR that was designed to improve sensitivity and specificity. Nested PCR involves the use of two primer sets and two successive PCR reactions. The first set of primers are designed to anneal to sequences upstream from the second set of primers and are used in an initial PCR reaction. Amplicons resulting from the first PCR reaction are used as template for a second set of primers and a second amplification step. Sensitivity and specificity of DNA amplification may be significantly enhanced with this technique. However, the potential for carryover contamination of the reaction is typically also increased due to additional manipulation of amplicon products. To minimize carryover, different parts of the process should be physically separated from one another, preferably in entirely separate rooms. Amplicons from nested PCR assays are detected in the same manner as in PCR above.”

The terms “polynucleotide” and “nucleic acid” (e.g., as they related to antibody sequences or antibody cDNA described herein) refer to a polymer composed of a multiplicity of nucleotide units (ribonucleotide or deoxyribonucleotide or related structural variants) linked via phosphodiester bonds. A polynucleotide or nucleic acid can be of substantially any length, typically from about six (6) nucleotides to about 10⁹ nucleotides or larger. Polynucleotides and nucleic acids include RNA, cDNA, genomic DNA. In particular, the polynucleotides and nucleic acids of the present invention refer to polynucleotides encoding a chromatin protein, a nucleotide modifying enzyme and/or fusion polypeptides of a chromatin protein and a nucleotide modifying enzyme, including mRNAs, DNAs, cDNAs, genomic DNA, and polynucleotides encoding fragments, derivatives and analogs thereof. Useful fragments and derivatives include those based on all possible codon choices for the same amino acid, and codon choices based on conservative amino acid substitutions. Useful derivatives further include those having at least 50% or at least 70% polynucleotide sequence identity, and more preferably 80%, still more preferably 90% sequence identity, to a native chromatin binding protein or to a nucleotide modifying enzyme.

The term “oligonucleotide” refers to a polynucleotide of from about six (6) to about one hundred (100) nucleotides or more in length. Thus, oligonucleotides are a subset of polynucleotides. Oligonucleotides can be synthesized manually, or on an automated oligonucleotide synthesizer (for example, those manufactured by Applied BioSystems (Foster City, Calif.)) according to specifications provided by the manufacturer or they can be the result of restriction enzyme digestion and fractionation.

The term “primer” as used herein refers to a polynucleotide, typically an oligonucleotide, whether occurring naturally, as in an enzyme digest, or whether produced synthetically, which acts as a point of initiation of polynucleotide synthesis when used under conditions in which a primer extension product is synthesized. A primer can be single-stranded or double-stranded. As described herein, in some aspects of the present disclosure, the primer or primers are immobilized within or on a microfluidic device such as a device described herein.

The terms “identical” or “percent identity,” in the context of two or more nucleic acids or polypeptide sequences, refer to two or more sequences or subsequences that are the same or have a specified percentage of nucleotides or amino acid residues that are the same, when compared and aligned for maximum correspondence, as measured using one of the following sequence comparison algorithms, or by visual inspection.

The phrase “substantially identical,” in the context of two nucleic acids or polypeptides, refers to two or more sequences or subsequences that have at least 60%, typically 80%, most typically 90-95% nucleotide or amino acid residue identity, when compared and aligned for maximum correspondence, as measured using one of the following sequence comparison algorithms, or by visual inspection. An indication that two polypeptide sequences are “substantially identical” is that one polypeptide is immunologically reactive with antibodies raised against the second polypeptide.

“Similarity” or “percent similarity” in the context of two nucleic acids or polypeptides, refer to two or more sequences or subsequences that are the same or have a specified percentage of nucleotides or amino acid residues or conservative substitutions thereof, that are the same, when compared and aligned for maximum correspondence, as measured using one of the following sequence comparison algorithms, or by visual inspection. By way of example, a first sequence can be considered similar to a second sequence when the first sequence is at least 30%, 40%, 50%, 60%, 70%, 75%, 80%, 90%, or even 95% identical, or conservatively substituted, to the second sequence when compared to an equal number of nucleotides or amino acids as the number contained in the first sequence, or when compared to an alignment that has been aligned by a computer similarity program known in the art, as discussed below.

Generally, other nomenclature used herein and many of the laboratory procedures in cell culture, molecular genetics and nucleic acid chemistry and hybridization, which are described below, are those well-known and commonly employed in the art. (See generally Ausubel et al. (1996) supra; Sambrook et al, Molecular Cloning: A Laboratory Manual, Second Edition, Cold Spring Harbor Laboratory Press, New York (1989), which are incorporated by reference herein). Standard techniques are used for recombinant nucleic acid methods, polynucleotide synthesis, preparation of biological samples, preparation of cDNA fragments, isolation of mRNA and the like. Generally enzymatic reactions and purification steps are performed according to the manufacturers' specifications.

In some embodiments of the present disclosure, method of cloning an antibody (or methods of producing an isolated antibody or methods of preparing an expression vector comprising an antibody) from a single cell are provided, comprising: a. isolating a single cell from a population of cells; b. separately amplifying a heavy chain complementary DNA (cDNA) and a light chain cDNA from said single cell, wherein each amplification comprise two polymerase chase reactions (PCR), wherein the first PCR reaction comprises an outer forward primer capable of specifically hybridizing to a barcode, and an outer reverse primer capable of specifically hybridizing to the antibody constant region, and wherein the second PCR reaction comprises an inner forward primer capable of specifically hybridizing to the 5′ end of the variable region of the antibody and an inner reverse primer capable of specifically hybridizing to the 3′ end of the variable region of the antibody; and c. inserting the amplified heavy chain cDNA and the amplified light chain cDNA of step (b) into separate vectors, thereby cloning an antibody from a single cell.

Of course, it will be appreciated from the disclosure herein that the compositions and methods provided herein comprise, in various embodiments, 1) Generating heavy and light chain cDNA and simultaneously attaching a unique barcode to each chain, which uniquely identifies the heavy and light chain cDNA from the single cell; 2) Pooling and amplifying the library of heavy and light chain cDNA; 3) Sequencing the heavy and light chain cDNA, as well as the unique barcodes; 4) Computationally identifying an antibody of interest based on the sequencing data; and 5) informatically designing primers to amplify the heavy and light chain cDNA from that single cell within the pooled library.

As used herein, the term “barcode” refers to refers to a nucleic acid sequence which uniquely or nearly uniquely identifies a nucleic acid molecule within a pool of molecules. Sequencing can reveal a certain barcode coupled to a nucleic acid molecule of interest. In some instances, the barcode can therefore allow identification, selection, or amplification of DNA molecules that are coupled thereto. (See, e.g., U.S. Pat. No. 10,155,942, incorporated by reference herein in its entirety).

Several strategies for uniquely barcoding individual cells are contemplated herein and are known in the art. In one exemplary strategy, the barcode is included in a nucleic acid that acts as a “template switching sequence” during reverse transcription (see, e.g., US20180105808A1, incorporated by reference in its entirety herein). In another strategy, the barcode is included in a PCR primer that primes on the 5′ end. In another strategy, the barcode is included in a PCR primer that primes on the 3′ end.

The sequences amplified from the single cell may, in various embodiments, be inserted or cloned into 1, 2, 3, 4, 5 or more vectors, e.g., expression vectors. For example, the heavy and light chain sequences may be cloned into the same vector or into separate vectors.

In some embodiments, the single cell is isolated from a pooled library of cells. According to various embodiments of the present disclosure, the pool of cells has been sequenced or otherwise engineered prior to the isolation of the single cell.

Various methods are contemplated by the present disclosure for the isolation and/or engineering (e.g., attaching a barcode) to the single cell. In some embodiments, the single cell is isolated by capturing the single cell in a droplet of oil using a microfluidic device. Single cells can be uniquely barcoded in other ways. One way is microfluidic capture in a chamber (see Fluidigm's Cl chip; e.g., A. R. Wu, et al., Nature Methods, 11, 41-46 (2014)). Another way is single-cell combinatorial indexing (which involves attaching several different barcodes, and their combination is unique) (See, e.g., J. Cao, et al., Science, 357 (6352):661-667 (2017)). Uniquely labeling single cells is summarized in G. Chen et al., Front. Genet., 2019, 10.3389/fgene.2019.00317. Each of the aforementioned publications are incorporated and contemplated herein.

In various aspects, a system such as the 10×Genomics Chromium Single Cell 5′ V(D)J system is used.

A “pool” or a “pool of cells” according to various embodiments may comprise 100, 1,000, 10, 000, 100,000 or more cells.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a conformation switching probe” includes a plurality of such conformation switching probes and reference to “the microfluidic device” includes reference to one or more microfluidic devices and equivalents thereof known to those skilled in the art, and so forth. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. This is intended to provide support for all such combinations.

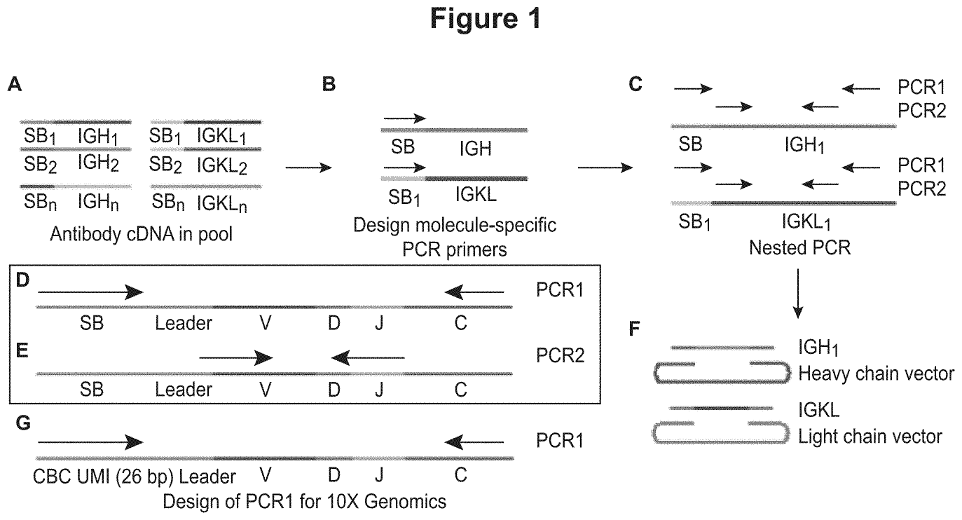

As disclosed herein, in various embodiments a strategy for cloning antibody heavy- and light-chain cDNA from a single B cell within a pooled library is provided by leveraging the unique sequence barcodes that are attached to molecules of cDNA during sample preparation. These sequence barcodes typically include a cell barcode (CBC) used to distinguish individual cells and a unique molecular identifier (UMI) used to distinguish individual molecules of template RNA (FIG. 1A). After sequencing, the antibody heavy- and light-chain sequences, and their corresponding sequence barcodes are known. These sequence barcodes are then used as molecular tags to retrieve cDNA from a single cell.

The present disclosure provides, in various embodiments, methods for cloning and expressing antibodies from single cells within pooled sequence libraries. Using primers that target the unique sequence barcodes attached to individual cDNA molecules during library preparation, a two-step nested PCR is performed to selectively amplify antibody cDNA from a single cell. This cDNA is then cloned via a one-step procedure into an expression vector for protein production and functional characterization.

The following materials and methods were used in in the Examples described herein.

Dataset

For computational analysis and experimental validation, a previously published dataset was used consisting of 94,259 single B cells (Horns et al., 2020). Briefly, the subject for this study was a female human aged 18 who was apparently healthy. Subject was vaccinated with the 2011-2012 seasonal trivalent inactivated influenza vaccine, and blood was collected by venipuncture 7 and 9 days afterwards (D7, D9), corresponding to the peak of the memory recall response (Horns et al., 2019). Peripheral blood mononuclear cells (PBMCs) were isolated using a Ficoll gradient and frozen according to Stanford Human Immune Monitoring Center protocol.

After thawing, B cells were magnetically enriched using B Cell Isolation Kit II (Miltenyi), then single cells were encapsulated in droplets using 16 lanes of the Chromium device (10×Genomics) with target loading of 14,000 cells per lane. Reverse transcription and cDNA amplification were performed using the Direct Enrichment protocol of the Single Cell 5′ V(D)J kit (10×Genomics). All steps were done according to manufacturer's instructions, except with additional cycles of PCR to obtain extra material for protocol testing (19 total cycles). Sequencing libraries were prepared using 50 ng of cDNA as input, then sequenced using the Illumina NextSeq 500 platform with paired-end reads of 150 bp each.

Antibody heavy- and light-chain transcripts were assembled for each cell using cellranger 2.1.0. Single B cells were identified by the presence of a single productive heavy chain and a single productive light chain, yielding a total of 94,259 single B cells.

Primer Design for SPAR

Primer design for SPAR consists of choosing nested PCR primers targeting the antibody gene of interest. In the 10×Genomics VDJ platform, each antibody sequence is typically assembled from sequencing reads from multiple cDNA molecules, which are tagged with the same cell barcode (CBC), but different unique molecular identifiers (UMIs). Accordingly, the gene can be addressed using a primer specific to the CBC and any of the UMIs. To design primers for PCR1, we first compiled a list of all UMIs supporting the antibody gene assembly, based on the output of cellranger 2.1.0. Full-length cDNA sequences were formed by concatenating partial read 1 primer, CBC, UMI, and template switch oligo (TSO) sequences to the assembled gene (Table 1). Position of the constant region was determined based on the annotation provided by cellranger 2.1.0. Primers were generated using Primer3 4.1.0 with default parameters except PRIMER_OPT_SIZE=26; PRIMER_MIN_SIZE=22; PRIMER_MAX_SIZE=35; PRIMER_OPT_TM=67; PRIMER_MIN_TM=53; PRIMER_MAX_TM=72; PRIMER_MIN_GC=30; PRIMER_MAX_GC=70; PRIMER_SALT_DIVALENT=2.5; PRIMER_DNA_CONC=200; PRIMER_PRODUCT_SIZE_RANGE=250-750. Target was specified as region bounded exclusively by the UMI and constant region, forcing primers to be selected within the CBC and UMI, and the constant region. Primers were allowed to include up to 5 bases of the partial read 1 sequence. Scores of primer pairs were aggregated across all UMIs and the best-scoring primer pair was accepted as the PCR1 primers.

Primers for PCR2 were designed that flank the antibody variable region. The amplicon sequence produced by PCR1 was determined, then located the variable region sequence using IgBlast (Ye et al., 2013, Nucleic Acids Research 41, W34-W40). Primers were generated using Primer3 4.1.0 with default parameters except PRIMER_OPT_SIZE=20; PRIMER_MIN_SIZE=13; PRIMER_MAX_SIZE=35; PRIMER_MIN_TM=50; PRIMER_OPT_TM=60; PRIMER_MAX_TM=70; PRIMER_MIN_GC=30; PRIMER_MAX_GC=70; PRIMER_SALT_DIVALENT=2.5; PRIMER_DNA_CONC=200; PRIMER_PRODUCT_SIZE_RANGE=100-700. The best-scoring primer pair was accepted as the PCR2 primers.

This workflow was implemented using custom Python scripts. For each individual cell, the workflow was carried out separately for the heavy- and light-chain genes.

TABLE 1

| | | SEQ |
|---|---|---|
| Name | Sequence (5′ to 3′) | ID NO: |
| Partial read 1 primer | CTACACGACGCTCTTCCGATCT | 1 |
| Template switch oligo | TTTCTTATATGGG | 2 |
| SPAR PCR1 F Cell 1 Heavy | ATCCGAATCGCATGGCCTCATAGTTA | 3 |
| SPAR PCR1 R Cell 1 Heavy | AGACGAGGGGGAAAAGGGTTGG | 4 |
| SPAR PCR2 F Cell 1 Heavy | CTAGTAGCAACTGCAACCGGTGTACATTCCCAGGTGCAGC | 5 |
| | TGC | |
| SPAR PCR2 R Cell 1 Heavy | GGGGAAGACCGATGGGCCCTTGGTCGACGCCTGAGGAGA | 6 |
| | CGGTGACCA | |
| SPAR PCR1 F Cell 1 Light | CTATCCGAATCGCATGGCACCTTAAC | 7 |
| SPAR PCR1 R Cell 1 Light | CTTGTTGGCTTGAAGCTCCTCAGAG | 8 |
| SPAR PCR2 F Cell 1 Light | CTAGTAGCAACTGCAACCGGTGTACATTCACAGTCTGCCC | 9 |
| | TGACTCAG | |
| SPAR PCR2 R Cell 1 Light | AGTGACCGAGGGGGCAGCCTTGGGCTGACCCTAGGACGGT | 10 |
| | CAGCTTGG | |
| SPAR PCR1 F Cell 2 Heavy | GACAGAGGTCGATTGTGCCGTACAC | 11 |
| SPAR PCR1 R Cell 2 Heavy | CTCACAGGAGACGAGGGGGAAAAG | 12 |
| SPAR PCR2 F Cell 2 Heavy | CTAGTAGCAACTGCAACCGGTGTACATTCCGGTGCAGCTG | 13 |
| | GTG | |
| SPAR PCR2 R Cell 2 Heavy | GGGGAAGACCGATGGGCCCTTGGTCGACGCCTGAGGAGA | 14 |
| | CGGTGACCA | |
| SPAR PCR1 F Cell 2 Light | GACAGAGGTCGATTGTCATCCGAAGT | 15 |
| SPAR PCR1 R Cell 2 Light | AGATGGCGGGAAGATGAAGACAGATG | 16 |
| SPAR PCR2 F Cell 2 Light | CTAGTAGCAACTGCAACCGGTGTACATTCAGACATCCAGA | 17 |
| | TGACCCAGTC | |
| SPAR PCR2 R Cell 2 Light | GATGAAGACAGATGGTGCAGCCACCGTACGGTTTGACCTC | 18 |
| | CACCCTGG | |
| SPAR PCR1 F Cell 3 Heavy | GCATGATCAAGCGTAGACAGCATGAA | 19 |
| SPAR PCR1 R Cell 3 Heavy | CTCACAGGAGACGAGGGGGAAAAG | 20 |
| SPAR PCR2 F Cell 3 Heavy | CTAGTAGCAACTGCAACCGGTGTACATTCCGAGGTGCGGC | 21 |
| | TGGTGGAGTC | |
| SPAR PCR2 R Cell 3 Heavy | GGGGAAGACCGATGGGCCCTTGGTCGACGCCTGAGGAGA | 22 |
| | CGGTGACCA | |
| SPAR PCR1 F Cell 3 Light | ATCTGCATGATCAAGCGTAGAAGC | 23 |
| SPAR PCR1 R Cell 3 Light | AGATTTCAACTGCTCATCAGATGGCG | 24 |
| SPAR PCR2 F Cell 3 Light | CTAGTAGCAACTGCAACCGGTGTACATTCAGATATTGTGA | 25 |
| | TGACTCAGTCTCC | |
| SPAR PCR2 R Cell 3 Light | GATGAAGACAGATGGTGCAGCCACCGTACGGTTTGATCTC | 26 |
| | CAGCTTGGTC | |
| SPAR PCR1 F Cell 4 Heavy | TCTGATCCTCCTCAATTGGGATTCCG | 27 |
| SPAR PCR1 R Cell 4 Heavy | CTCACAGGAGACGAGGGGGAAAAG | 28 |
| SPAR PCR2 F Cell 4 Heavy | CAGGTGCAGCTGC | 29 |
| SPAR PCR2 R Cell 4 Heavy | GGAGACGGTGACCA | 30 |
| SPAR PCR1 F Cell 4 Light | ATCCTCCTCAATTGCTCGCGTCT | 31 |
| SPAR PCR1 R Cell 4 Light | GAAGATGAAGACAGATGGTGCAGCC | 32 |
| SPAR PCR2 F Cell 4 Light | GATATTGTGATGACCCAGACTC | 33 |
| SPAR PCR2 R Cell 4 Light | TCTCCAGCTTGGTC | 34 |
| SPAR PCR1 F Cell 5 Heavy | TCTATCTGCCGTCTCTTATACTGCGC | 35 |
| SPAR PCR1 R Cell 5 Heavy | GAAGACCGATGGGCCCTTGGTG | 36 |
| SPAR PCR2 F Cell 5 Heavy | CAGGTGCAGCTGGTG | 37 |
| SPAR PCR2 R Cell 5 Heavy | AGAGACGGTGACCATT | 38 |
| SPAR PCR1 F Cell 5 Light | TCTATCTGCCGTCTCTTATCGTGCTG | 39 |
| SPAR PCR1 R Cell 5 Light | AGATTTCAACTGCTCATCAGATGGCG | 40 |
| SPAR PCR2 F Cell 5 Light | GACATCCAGATGACGCAG | 41 |
| SPAR PCR2 R Cell 5 Light | TTTCCACCTTGGTCC | 42 |
| SPAR PCR1 F Cell 6 Heavy | ATTTCTGGTTGATTCGTCACGTCCAC | 43 |
| SPAR PCR1 R Cell 6 Heavy | CAGAGGCTCAGCGGGAAGACCTTG | 44 |
| SPAR PCR2 F Cell 6 Heavy | GAGGTGCAGCTGTTGGA | 45 |
| SPAR PCR2 R Cell 6 Heavy | CTGAGGAGACGGTGACCA | 46 |
| SPAR PCR1 F Cell 6 Light | TCTATTTCTGGTTGATTCGACTCACG | 47 |
| SPAR PCR1 R Cell 6 Light | AGATTTCAACTGCTCATCAGATGGCG | 48 |
| SPAR PCR2 F Cell 6 Light | GAAATTGTGTTGACGCAG | 49 |
| SPAR PCR2 R Cell 6 Light | GTTTAATCTCCAGTCGTGTCC | 50 |
| SPAR PCR1 F Cell 7 Heavy | GCCAATCTGCGACGCAAACAGTTT | 51 |
| SPAR PCR1 R Cell 7 Heavy | CTCACAGGAGACGAGGGGGAAAAG | 52 |
| SPAR PCR2 F Cell 7 Heavy | GAGGTGCAGCTGGTGG | 53 |
| SPAR PCR2 R Cell 7 Heavy | CTGAGGAGACGGTGACCA | 54 |
| SPAR PCR1 F Cell 7 Light | CAATCTGCGACGCTCAGGTTGG | 55 |
| SPAR PCR1 R Cell 7 Light | CACACAACAGAGGCAGTTCCAGATTT | 56 |
| SPAR PCR2 F Cell 7 Light | GACATCCAGATGACCCAGTC | 57 |
| SPAR PCR2 R Cell 7 Light | GTTTGATCTCCAGCTTGGTC | 58 |
| SPAR PCR1 F Cell 8 Heavy | TGGCTGGTCAATGAATGAAGCAAACA | 59 |
| SPAR PCR1 R Cell 8 Heavy | CTCACAGGAGACGAGGGGGAAAAG | 60 |
| SPAR PCR2 F Cell 8 Heavy | CAGGTGCAGCTGGTG | 61 |
| SPAR PCR2 R Cell 8 Heavy | TGAGGAGACGGTGACC | 62 |
| SPAR PCR1 F Cell 8 Light | GATCTGGCTGGTCAATGAATGGGAC | 63 |
| SPAR PCR1 R Cell 8 Light | AGATTTCAACTGCTCATCAGATGGCG | 64 |
| SPAR PCR2 F Cell 8 Light | GAAATAGTGATGACGCAG | 65 |
| SPAR PCR2 R Cell 8 Light | GTTTGATATCCACTTTGGTCCC | 66 |
| SPAR Vector F Cell 1 | GCAGCTGCACCTGGGAATGTACACCGGTTGCAGTTGCTAC | 67 |
| Heavy | TAG | |
| SPAR Vector R Cell 1 | TGGTCACCGTCTCCTCAGGCGTCGACCAAGGGCCCATCGG | 68 |
| Heavy | TCTTCCCC | |
| SPAR Vector F Cell 1 | CTGAGTCAGGGCAGACTGTGAATGTACACCGGTTGCAGTT | 69 |
| Light | GCTACTAG | |
| SPAR Vector R Cell 1 | CCAAGCTGACCGTCCTAGGGTCAGCCCAAGGCTGCCCCCT | 70 |
| Light | CGGTCACT | |
| SPAR Vector F Cell 2 | CACCAGCTGCACCGGAATGTACACCGGTTGCAGTTGCTAC | 71 |
| Heavy | TAG | |
| SPAR Vector R Cell 2 | TGGTCACCGTCTCCTCAGGCGTCGACCAAGGGCCCATCGG | 72 |
| Heavy | TCTTCCCC | |
| SPAR Vector F Cell 2 | GACTGGGTCATCTGGATGTCTGAATGTACACCGGTTGCAG | 73 |
| Light | TTGCTACTAG | |
| SPAR Vector R Cell 2 | CCAGGGTGGAGGTCAAACCGTACGGTGGCTGCACCATCTG | 74 |
| Light | TCTTCATC | |
| SPAR Vector F Cell 3 | GACTCCACCAGCCGCACCTCGGAATGTACACCGGTTGCAG | 75 |
| Heavy | TTGCTACTAG | |
| SPAR Vector R Cell 3 | TGGTCACCGTCTCCTCAGGCGTCGACCAAGGGCCCATCGG | 76 |
| Heavy | TCTTCCCC | |
| SPAR Vector F Cell 3 | GGAGACTGAGTCATCACAATATCTGAATGTACACCGGTTG | 77 |
| Light | CAGTTGCTACTAG | |
| SPAR Vector R Cell 3 | GACCAAGCTGGAGATCAAACCGTACGGTGGCTGCACCATC | 78 |
| Light | TGTCTTCATC | |

Computational Analysis of Retrievability of Human Antibody Repertoire

To assess how much of the human antibody repertoire can be retrieved, SPAR primer design was performed for all single cells in our dataset (n=94,259). The default parameters for SPAR primer design described herein were used. An antibody was defined as retrievable if acceptable primers were found for PCR1 and PCR2 for both heavy- and light-chain genes. To assess the sequence similarity between PCR1 forward primers, the edit distance, also known as the Levenshtein distance, was calculated between all pairs of acceptable PCR1 forward primers for one pooled library (chosen at random). Predicted melting temperatures were calculated using Primer3. Data visualization and analysis were performed using JupyterLab (Kluyver et al., 2016, In Positioning and Power in Academic Publishing: Players, Agents and Agendas, F. Loizides, and B. Scmidt, eds. (IOS Press), pp. 87-90).

Experimental Validation of SPAR

To demonstrate that SPAR enables retrieval of single-cell antibody cDNA from pooled libraries, 8 cells were chosen at random from our dataset. For each cell, SPAR primers designed using the above workflow were synthesized (IDT).

PCR1 was performed using 12.5 uL of HiFi ReadyMix 2×(Kapa Biosystems), 0.75 uL each of forward and reverse primer (final concentration 0.3 uM each), 1 uL of template, and 10 uL of water. Template was 0.5 ng of cDNA from single-cell sequencing library preparation. PCR1 protocol was 95° C. for 3 min; 15 cycles of 98° C. for 20 sec, 65° C. for 15 sec, 72° C. for 1 min; 72° C. for 1 min. Primers from PCR1 were then degraded by adding 5 uL of PCR1 product to 2 uL of ExoSAP-IT (ThermoFisher), and incubating at 37° C. for 15 min, then 80° C. for 15 min.

PCR2 was performed using the same conditions, except using 1 uL of previous product as template. PCR2 protocol was 95° C. for 3 min; 15 cycles of 98° C. for 20 sec, 51° C. for 15 sec, 72° C. for 1 min; 72° C. for 1 min. Products were visualized by electrophoresis using E-Gel EX 2% agarose gels (ThermoFisher).

To demonstrate one-step cloning of SPAR products into expression vectors and verify their sequences, the PCR2 products were cloned using Gibson assembly and performed Sanger sequencing. Heavy- and kappa-chain expression vectors were VRC01 CMV/R HC and VRC01 CMV/R LC (Wu et al., 2010), respectively. Lambda-chain expression vector was VRC01 CMV/R Lambda LC, which was created by replacing the kappa constant region with the lambda constant region in VRC01 CMV/R LC (Wu et al., 2010, Science 329, 856-861). The reagent was obtained through the NIH AIDS Reagent Program, Division of AIDS, NIAID, NIH: CMVR VRC01 H/L, from Dr. John Mascola. Vectors were linearized by PCR. PCR conditions were the same as above, except 35 cycles were performed with annealing at 70° C., extension for 6 min, and final extension for 6 min. Template was 1 ng of vector. Products were purified using Ampure XP beads (Agencourt) at 0.7 bead to product volume ratio. Gibson assembly was performed using 10 uL of Gibson Assembly Master Mix (NEB), ˜150 fmol insert, and ˜50 fmol vector in a total volume of 20 uL. Products were transformed into E. cloni 10G Supreme cells (Lucigen) by electroporation following manufacturer's instruction. After overnight growth, eight colonies were picked and cultured in LB with 50 ug/mL kanamycin for 2 hours. Templates for colony PCR were prepared by diluting 10 uL of culture in 90 uL of water, then incubating at 95° C. for 10 min. Colony PCR was performed using 12.5 uL of HiFi ReadyMix 2× (Kapa Biosystems), 0.75 uL each of forward and reverse sequencing primers (SeqF and SeqHR, SeqLR, or SeqKR; final concentration 0.3 uM each), 1 uL of template, and 10 uL of water. PCR protocol was 95° C. for 3 min; 35 cycles of 98° C. for 20 sec, 55° C. for 15 sec, 72° C. for 1 min; 72° C. for 1 m. Sanger sequencing was performed on products using sequencing primers (Molecular Cloning Laboratories).

### Example 1

### Design of Selective PCR for Antibody Retrieval (SPAR)

A strategy was designed for selective amplification of target cDNA molecules using PCR primers that specifically bind sequence barcodes (FIG. 1B). The strategy uses nested PCR consisting of two steps (FIG. 1C). In the first step (PCR1), an outer forward primer was used that spans the sequence barcode, together with an outer reverse primer within the antibody constant region (FIG. 1D). In the second step (PCR2), an inner forward primer targeting the 5′ end of the variable region and an inner reverse primer targeting the 3′ end of the variable region was used (FIG. 1E). Importantly, the antibody complementarity determining region 3 (CDR3), located near the 3′ end of the variable region, is a hypervariable region that has high diversity within the antibody repertoire, allowing PCR2 primers targeting this region to enhance specificity. PCR2 yields full-length antibody variable region cDNA as a product. The PCR2 primers were designed with 5′ arms that are homologous to the expression vector, enabling a simple one-step cloning procedure (FIG. 1F).

This strategy was implemented, as one embodiment, using the 10×Genomics Chromium Single Cell 5′ V(D)J platform. This platform uses a 16 base pair (bp) CBC and 10 bp UMI. After single-cell paired heavy- and light-chain sequencing, the complete heavy- and light-chain variable region sequences, and the corresponding CBC and UMI sequences for each cDNA molecule are known. Forward PCR1 primers were designed to target this combined 26 bp CBC and UMI sequence (FIG. 1G). By performing computational primer design using Primer3 (Untergasser et al., 2012, Nucleic Acids Res. 40, e115), reverse PCR1 primers and PCR2 primer pairs were identified that were compatible with these forward PCR1 primers and had high annealing temperature (optimally 67° C.) to ensure specific amplification.

This strategy was tested using a dataset consisting of sequences and material from a previous study (Horns et al., 2020). This dataset consisted of 94,259 natively paired antibody heavy- and light-chain sequences obtained from single B cells, which were isolated from human peripheral blood at 7 or 9 days after influenza vaccination. Single-cell paired heavy-light chain antibody repertoire sequencing was performed using the 10×Genomics Chromium Single Cell 5′ V(D)J platform. Cells were pooled into 16 libraries, each having an average of 5,891 single cells (n=5,891±1,669, mean±s.d., range 298-7,169 cells). For experimental validation, the full-length cDNA pools generated by the standard sample preparation procedure were used.

### Example 2

### SPAR Primers can be Designed to Retrieve Most of the Human Antibody Repertoire

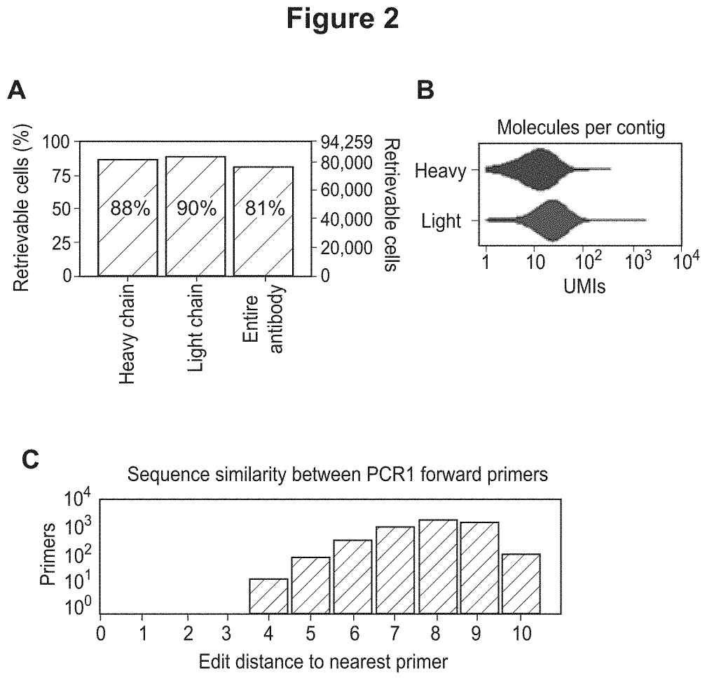

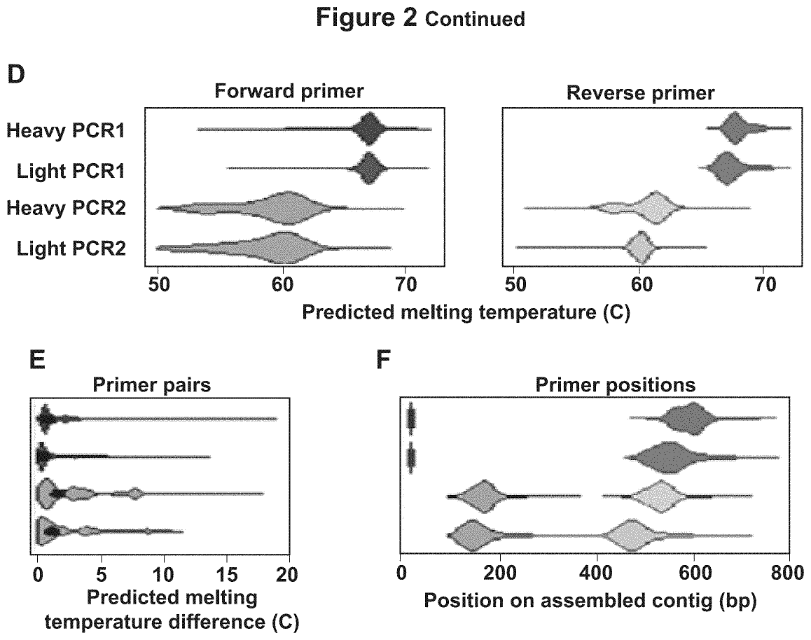

To assess how much of the human antibody repertoire can be retrieved using SPAR, SPAR primers were computationally designed to retrieve antibodies from all 94,259 single cells in the dataset. Successful primer design is a necessary condition for antibody retrieval. Overall, it was found that SPAR primers can be designed for 81% of these cells (FIG. 2A). At the single-chain level, SPAR primers can be designed for 88% of heavy-chain genes and 90% of light-chain genes (FIG. 2A). Because nearly all heavy- and light-chain genes were assembled based on sequencing of multiple molecules of cDNA, most antibodies can be addressed using multiple unique barcodes (FIG. 2B; median 11 UMIs per heavy chain, 21 per light chain), improving the likelihood of having at least one suitable PCR1 primer pair. These results indicate that SPAR primers can be designed to retrieve most antibodies in the human repertoire.

To assess the specificity of PCR1 primers targeting the sequence barcode, the similarity between PCR1 forward primers for heavy-chain genes in one pooled library was examined. It was found that PCR1 forward primer sequences are substantially dissimilar (FIG. 2C): the nearest sequence is 8 edits away on average, and the most similar pair of PCR1 forward primers is separated by 4 edits. This feature suggests that PCR1 primers are sufficiently divergent to support selective amplification.

SPAR primers have favorable properties for PCR. Predicted melting temperatures of PCR1 primers are high (FIG. 2D; 67.3±1.1 C, mean±s.d.) and well matched within pairs (FIG. 2E; temperature difference 1.1±1.3 C, mean±s.d.). PCR2 primer pairs have greater variation in predicted melting temperature due to stronger constraints on primer position (FIG. 2D; 59.4±2.4 C, mean±s.d.), but nevertheless have well matched melting temperature within pairs (FIG. 2E; temperature difference 2.5±2.6 C, mean±s.d.). PCR2 primers flank the variable region (FIG. 2F), permitting one-step cloning into expression vectors. These features indicate that SPAR primers support efficient selective PCR.

### Example 3

### SPAR Retrieves Full-Length Antibody Variable Region cDNA from Single Cells

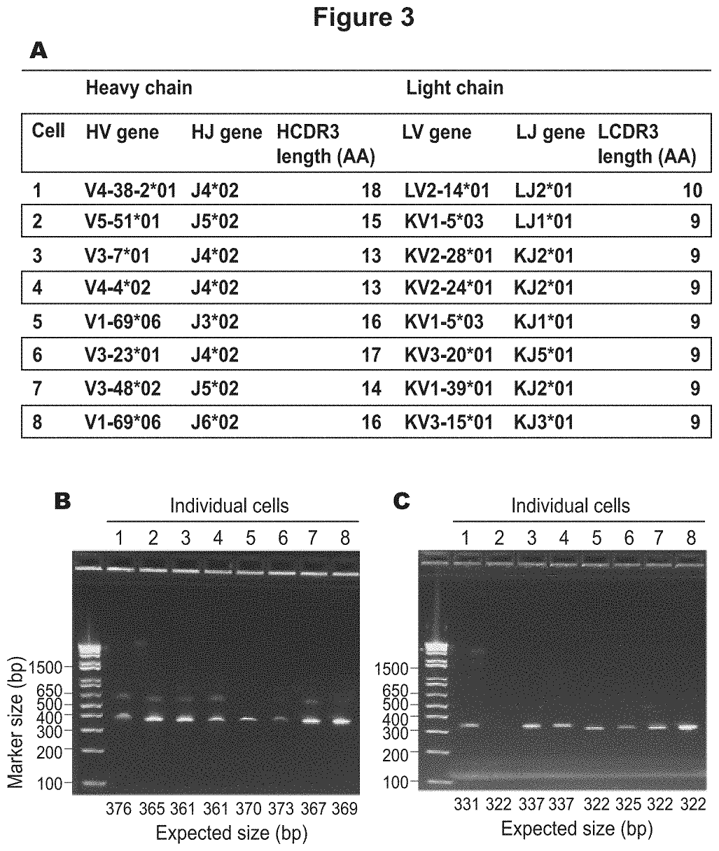

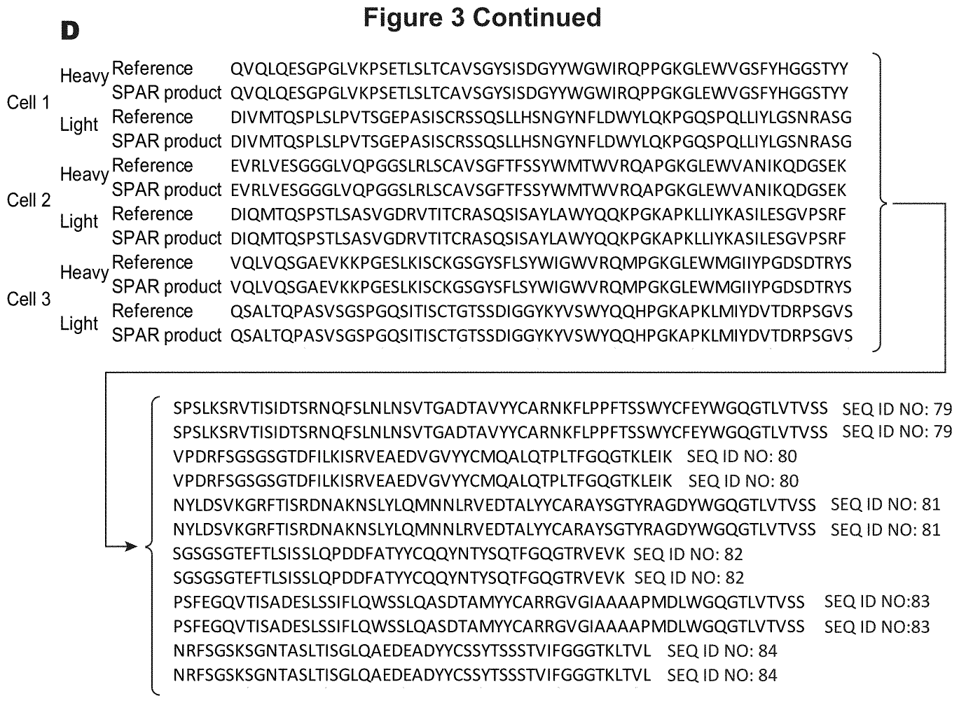

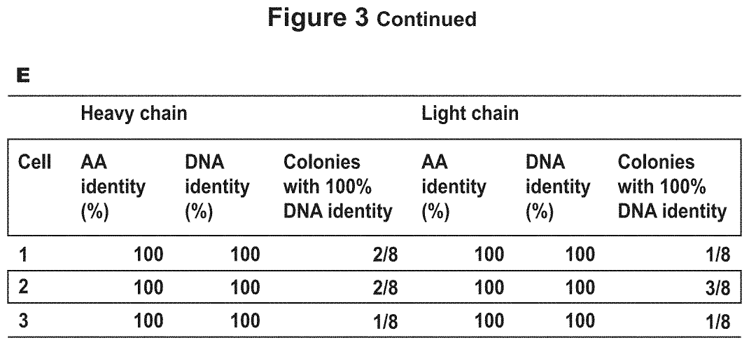

To experimentally test retrieval of antibody cDNA, SPAR was performed on 8 target cells chosen at random from the dataset. Agarose gel electrophoresis of the SPAR PCR2 products revealed successful retrieval of the expected, full-length antibody heavy- and light-chain variable region cDNA for all 8 targets (100%) (FIG. 3B and FIG. 3C). The cDNA for three of these antibodies was then cloned into heavy- and light-chain expression vectors using Gibson assembly. Using Sanger sequencing, it was confirmed that the target heavy- and light-chain sequences were retrieved for all three cells (FIG. 3D). In every case, the retrieved heavy- and light-chain cDNA had perfect (100%) amino acid and DNA identity to the target (FIG. 3E). These findings demonstrate that SPAR can retrieve full-length antibody cDNA from single cells within pooled libraries of ˜5,000 cells with high fidelity and efficiency.

In view of the above disclosure and examples, SPAR enables a simple workflow for cloning and expression of human antibodies for downstream functional characterization. After surveying antibody sequences at high-throughput using a single-cell sequencing approach, target antibodies can be chosen based on sequence or clonal characteristics, or single-cell phenotypes, such as transcriptome profile (Horns et al., 2020). Using SPAR, these antibodies can be cloned and expressed directly from the pooled cDNA library. As disclosed herein, >80% of human antibodies can be retrieved by SPAR. Notably, PCR-based mutagenesis could be used to generate variants in sequence space near these antibodies. SPAR costs ˜$70 per antibody, which is cheaper than or similar in price to gene synthesis. Importantly, SPAR can be performed within ˜29 hours, which is faster than the several-week turnaround time of gene synthesis. The speed of SPAR may be advantageous in scenarios requiring rapid response, such as antibody discovery for treatment of emerging infectious disease. Thus, SPAR enables rapid, low-cost expression of native antibodies for functional assays from pooled sequencing libraries.

To improve specificity, in certain embodiments the primer design algorithm could explicitly model and penalize possible mispriming within the cDNA pool. To further improve specificity and efficiency, in other embodiments the single-cell sequencing library preparation procedure could be modified to incorporate a longer sequence barcode. Similar barcoding schemes are used in other single-cell sequencing approaches, such as Drop-seq (Macosko et al., 2015, Cell 161, 1202-1214), Microwell-seq (Han et al., 2018, Cell 172, 1091-1107.e17), and SPLiT-seq (Rosenberg et al., 2018, Science 360, 176-182), and these are also amenable to our approach.

SPAR builds upon previous tag-directed retrieval methods for gene synthesis (Schwartz et al., 2012, Nature Methods 9, 913-915; and Woodruff et al., 2017, Nucleic Acids Res 45, 1553-1565) and enrichment of transcriptomes (Ranu et al., 2019). The exceptional diversity of natural antibody sequences (Briney et al., 2019, Nature 566, 393-397) enables highly specific nested PCR, permitting retrieval of individual cDNA molecules originating from single cells. The methods disclosed herein will facilitate biophysical characterization of antibodies, accelerating antibody discovery and enhancing our understanding of the relationship between antibody sequence and function.

The various embodiments described above can be combined to provide further embodiments. All U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

What is claimed is:

1. A method of cloning an antibody from a single cell comprising:
a. isolating a single cell from a population of cells;
b. separately amplifying a heavy chain complementary DNA (cDNA) and a light chain cDNA from said single cell, wherein each amplification comprise two polymerase chain reactions (PCR), wherein the first PCR reaction comprises an outer forward primer capable of specifically hybridizing to a barcode, and an outer reverse primer capable of specifically hybridizing to the antibody constant region, and wherein the second PCR reaction comprises an inner forward primer capable of specifically hybridizing to the 5′ end of the variable region of the antibody and an inner reverse primer capable of specifically hybridizing to the 3′ end of the variable region of the antibody; and
c. inserting the amplified heavy chain cDNA and the amplified light chain cDNA of step (b) into separate vectors, thereby cloning an antibody from a single cell.

2. The method of claim 1 wherein the two PCR reactions comprise a nested PCR process.

3. The method of claim 1 wherein the inner reverse primer hybridizes to a region of the 3′ end of the variable region comprising CDR3.

4. The method of claim 1 wherein the single cell is isolated from a pooled library of cells.

5. The method of claim 4 wherein the pooled library of cells comprise B cells.

6. The method of claim 5 wherein each B cell comprise a unique barcode adjacent or near the 5′ end of an antibody heavy chain and an antibody light chain cDNA.

7. The method of claim 6 wherein the barcode is approximately 5-50 nucleotides in length

8. The method of any one of claims 4-7 wherein the single cell is isolated by capturing the single cell in a droplet of aqueous solution using a microfluidic device.

9. The method of any one of claims 1-8 wherein the amplified cDNA comprises a full-length variable region cDNA.

10. The method of any one of claims 1-9 wherein the vector is an expression vector.

11. The method of claim 10 further comprising introducing the vector into a cell and expressing a full-length antibody comprising a variable region and a constant region.

12. The method of claim 11 further comprising purifying the antibody.

13. The method of claim 4 wherein the single cell is isolated from a sample from a subject.

14. The method of claim 13 wherein the sample is a peripheral blood sample, and wherein the single cell is isolated by capturing the single cell in a droplet of aqueous solution using a microfluidic device.

15. The method of any one of claims 13-14 wherein the sample is taken from a human subject suffering from a disease or disorder or following an infection or exposure to an antigen.

16. A method of preparing an expression vector comprising an antibody sequence from a single cell comprising:
a. isolating a single cell from a population of cells;
b. separately amplifying a heavy chain complementary DNA (cDNA) and a light chain cDNA from said single cell, wherein each amplification comprise two polymerase chase reactions (PCR), wherein the first PCR reaction comprises an outer forward primer capable of specifically hybridizing to a barcode, and an outer reverse primer capable of specifically hybridizing to the antibody constant region, and wherein the second PCR reaction comprises an inner forward primer capable of specifically hybridizing to the 5′ end of the variable region of the antibody and an inner reverse primer capable of specifically hybridizing to the 3′ end of the variable region of the antibody; and
c. inserting the amplified heavy chain cDNA and the amplified light chain cDNA of step (b) into separate vectors.

17. A method of preparing an antibody from a single cell comprising:
a. isolating a single cell from a population of cells;
b. separately amplifying a heavy chain complementary DNA (cDNA) and a light chain cDNA from said single cell, wherein each amplification comprise two polymerase chase reactions (PCR), wherein the first PCR reaction comprises an outer forward primer capable of specifically hybridizing to a barcode, and an outer reverse primer capable of specifically hybridizing to the antibody constant region, and wherein the second PCR reaction comprises an inner forward primer capable of specifically hybridizing to the 5′ end of the variable region of the antibody and an inner reverse primer capable of specifically hybridizing to the 3′ end of the variable region of the antibody;
c. inserting the amplified heavy chain cDNA and the amplified light chain cDNA of step (b) into separate vectors; and
d. expressing the heavy chain cDNA and light chain cDNA from the vectors in step (c) in a host cell under conditions that allow the production of an antibody.


SEQUENCE LISTING (84 sequences)

SEQ ID NO: 1 - DNA, 22 bp, Artificial Sequence
  Synthetic Polynucleotide
ctacacgacgctcttccgatct

SEQ ID NO: 2 - DNA, 13 bp, Artificial Sequence
  Synthetic Polynucleotide
tttcttatatggg

SEQ ID NO: 3 - DNA, 26 bp, Artificial Sequence
  Synthetic Polynucleotide
atccgaatcgcatggcctcatagtta

SEQ ID NO: 4 - DNA, 22 bp, Artificial Sequence
  Synthetic Polynucleotide
agacgagggggaaaagggttgg

SEQ ID NO: 5 - DNA, 43 bp, Artificial Sequence
  Synthetic Polynucleotide
ctagtagcaactgcaaccggtgtacattcccaggtgcagctgc

SEQ ID NO: 6 - DNA, 48 bp, Artificial Sequence
  Synthetic Polynucleotide
ggggaagaccgatgggcccttggtcgacgcctgaggagacggtgacca

SEQ ID NO: 7 - DNA, 26 bp, Artificial Sequence
  Synthetic Polynucleotide
ctatccgaatcgcatggcaccttaac

SEQ ID NO: 8 - DNA, 25 bp, Artificial Sequence
  Synthetic Polynucleotide
cttgttggcttgaagctcctcagag

SEQ ID NO: 9 - DNA, 48 bp, Artificial Sequence
  Synthetic Polynucleotide
ctagtagcaactgcaaccggtgtacattcacagtctgccctgactcag

SEQ ID NO: 10 - DNA, 48 bp, Artificial Sequence
  Synthetic Polynucleotide
agtgaccgagggggcagccttgggctgaccctaggacggtcagcttgg

SEQ ID NO: 11 - DNA, 25 bp, Artificial Sequence
  Synthetic Polynucleotide
gacagaggtcgattgtgccgtacac

SEQ ID NO: 12 - DNA, 24 bp, Artificial Sequence
  Synthetic Polynucleotide
ctcacaggagacgagggggaaaag

SEQ ID NO: 13 - DNA, 43 bp, Artificial Sequence
  Synthetic Polynucleotide
ctagtagcaactgcaaccggtgtacattccggtgcagctggtg

SEQ ID NO: 14 - DNA, 48 bp, Artificial Sequence; identical to SEQ ID NO: 6
  Synthetic Polynucleotide

SEQ ID NO: 15 - DNA, 26 bp, Artificial Sequence
  Synthetic Polynucleotide
gacagaggtcgattgtcatccgaagt

SEQ ID NO: 16 - DNA, 26 bp, Artificial Sequence
  Synthetic Polynucleotide
agatggcgggaagatgaagacagatg

SEQ ID NO: 17 - DNA, 50 bp, Artificial Sequence
  Synthetic Polynucleotide
ctagtagcaactgcaaccggtgtacattcagacatccagatgacccagtc

SEQ ID NO: 18 - DNA, 48 bp, Artificial Sequence
  Synthetic Polynucleotide
gatgaagacagatggtgcagccaccgtacggtttgacctccaccctgg

SEQ ID NO: 19 - DNA, 26 bp, Artificial Sequence
  Synthetic Polynucleotide
gcatgatcaagcgtagacagcatgaa

SEQ ID NO: 20 - DNA, 24 bp, Artificial Sequence
  Synthetic Polynucleotide
ctcacaggagacgagggggaaaag

SEQ ID NO: 21 - DNA, 50 bp, Artificial Sequence
  Synthetic Polynucleotide
ctagtagcaactgcaaccggtgtacattccgaggtgcggctggtggagtc

SEQ ID NO: 22 - DNA, 48 bp, Artificial Sequence; identical to SEQ ID NO: 6
  Synthetic Polynucleotide

SEQ ID NO: 23 - DNA, 24 bp, Artificial Sequence
  Synthetic Polynucleotide
atctgcatgatcaagcgtagaagc

SEQ ID NO: 24 - DNA, 26 bp, Artificial Sequence
  Synthetic Polynucleotide
agatttcaactgctcatcagatggcg

SEQ ID NO: 25 - DNA, 53 bp, Artificial Sequence
  Synthetic Polynucleotide
ctagtagcaactgcaaccggtgtacattcagatattgtgatgactcagtctcc

SEQ ID NO: 26 - DNA, 50 bp, Artificial Sequence
  Synthetic Polynucleotide
gatgaagacagatggtgcagccaccgtacggtttgatctccagcttggtc

SEQ ID NO: 27 - DNA, 26 bp, Artificial Sequence
  Synthetic Polynucleotide
tctgatcctcctcaattgggattccg

SEQ ID NO: 28 - DNA, 24 bp, Artificial Sequence
  Synthetic Polynucleotide
ctcacaggagacgagggggaaaag

SEQ ID NO: 29 - DNA, 13 bp, Artificial Sequence
  Synthetic Polynucleotide
caggtgcagctgc

SEQ ID NO: 30 - DNA, 14 bp, Artificial Sequence
  Synthetic Polynucleotide
ggagacggtgacca

SEQ ID NO: 31 - DNA, 23 bp, Artificial Sequence
  Synthetic Polynucleotide
atcctcctcaattgctcgcgtct

SEQ ID NO: 32 - DNA, 25 bp, Artificial Sequence
  Synthetic Polynucleotide
gaagatgaagacagatggtgcagcc

SEQ ID NO: 33 - DNA, 22 bp, Artificial Sequence
  Synthetic Polynucleotide
gatattgtgatgacccagactc

SEQ ID NO: 34 - DNA, 14 bp, Artificial Sequence
  Synthetic Polynucleotide
tctccagcttggtc

SEQ ID NO: 35 - DNA, 26 bp, Artificial Sequence
  Synthetic Polynucleotide
tctatctgccgtctcttatactgcgc

SEQ ID NO: 36 - DNA, 22 bp, Artificial Sequence
  Synthetic Polynucleotide
gaagaccgatgggcccttggtg

SEQ ID NO: 37 - DNA, 15 bp, Artificial Sequence
  Synthetic Polynucleotide
caggtgcagctggtg

SEQ ID NO: 38 - DNA, 16 bp, Artificial Sequence
  Synthetic Polynucleotide
agagacggtgaccatt

SEQ ID NO: 39 - DNA, 26 bp, Artificial Sequence
  Synthetic Polynucleotide
tctatctgccgtctcttatcgtgctg

SEQ ID NO: 40 - DNA, 26 bp, Artificial Sequence
  Synthetic Polynucleotide
agatttcaactgctcatcagatggcg

SEQ ID NO: 41 - DNA, 18 bp, Artificial Sequence
  Synthetic Polynucleotide
gacatccagatgacgcag

SEQ ID NO: 42 - DNA, 15 bp, Artificial Sequence
  Synthetic Polynucleotide
tttccaccttggtcc

SEQ ID NO: 43 - DNA, 26 bp, Artificial Sequence
  Synthetic Polynucleotide
atttctggttgattcgtcacgtccac

SEQ ID NO: 44 - DNA, 24 bp, Artificial Sequence
  Synthetic Polynucleotide
cagaggctcagcgggaagaccttg

SEQ ID NO: 45 - DNA, 17 bp, Artificial Sequence
  Synthetic Polynucleotide
gaggtgcagctgttgga

SEQ ID NO: 46 - DNA, 18 bp, Artificial Sequence
  Synthetic Polynucleotide
ctgaggagacggtgacca

SEQ ID NO: 47 - DNA, 26 bp, Artificial Sequence
  Synthetic Polynucleotide
tctatttctggttgattcgactcacg

SEQ ID NO: 48 - DNA, 26 bp, Artificial Sequence
  Synthetic Polynucleotide
agatttcaactgctcatcagatggcg

SEQ ID NO: 49 - DNA, 18 bp, Artificial Sequence
  Synthetic Polynucleotide
gaaattgtgttgacgcag

SEQ ID NO: 50 - DNA, 21 bp, Artificial Sequence
  Synthetic Polynucleotide
gtttaatctccagtcgtgtcc

SEQ ID NO: 51 - DNA, 24 bp, Artificial Sequence
  Synthetic Polynucleotide
gccaatctgcgacgcaaacagttt

SEQ ID NO: 52 - DNA, 24 bp, Artificial Sequence
  Synthetic Polynucleotide
ctcacaggagacgagggggaaaag

SEQ ID NO: 53 - DNA, 16 bp, Artificial Sequence
  Synthetic Polynucleotide
gaggtgcagctggtgg

SEQ ID NO: 54 - DNA, 18 bp, Artificial Sequence
  Synthetic Polynucleotide
ctgaggagacggtgacca

SEQ ID NO: 55 - DNA, 22 bp, Artificial Sequence
  Synthetic Polynucleotide
caatctgcgacgctcaggttgg

SEQ ID NO: 56 - DNA, 26 bp, Artificial Sequence
  Synthetic Polynucleotide
cacacaacagaggcagttccagattt

SEQ ID NO: 57 - DNA, 20 bp, Artificial Sequence
  Synthetic Polynucleotide
gacatccagatgacccagtc

SEQ ID NO: 58 - DNA, 20 bp, Artificial Sequence
  Synthetic Polynucleotide
gtttgatctccagcttggtc

SEQ ID NO: 59 - DNA, 26 bp, Artificial Sequence
  Synthetic Polynucleotide
tggctggtcaatgaatgaagcaaaca

SEQ ID NO: 60 - DNA, 24 bp, Artificial Sequence
  Synthetic Polynucleotide
ctcacaggagacgagggggaaaag

SEQ ID NO: 61 - DNA, 15 bp, Artificial Sequence
  Synthetic Polynucleotide
caggtgcagctggtg

SEQ ID NO: 62 - DNA, 16 bp, Artificial Sequence
  Synthetic Polynucleotide
tgaggagacggtgacc

SEQ ID NO: 63 - DNA, 25 bp, Artificial Sequence
  Synthetic Polynucleotide
gatctggctggtcaatgaatgggac

SEQ ID NO: 64 - DNA, 26 bp, Artificial Sequence
  Synthetic Polynucleotide
agatttcaactgctcatcagatggcg

SEQ ID NO: 65 - DNA, 18 bp, Artificial Sequence
  Synthetic Polynucleotide
gaaatagtgatgacgcag

SEQ ID NO: 66 - DNA, 22 bp, Artificial Sequence
  Synthetic Polynucleotide
gtttgatatccactttggtccc

SEQ ID NO: 67 - DNA, 43 bp, Artificial Sequence
  Synthetic Polynucleotide
gcagctgcacctgggaatgtacaccggttgcagttgctactag

SEQ ID NO: 68 - DNA, 48 bp, Artificial Sequence
  Synthetic Polynucleotide
tggtcaccgtctcctcaggcgtcgaccaagggcccatcggtcttcccc

SEQ ID NO: 69 - DNA, 48 bp, Artificial Sequence
  Synthetic Polynucleotide
ctgagtcagggcagactgtgaatgtacaccggttgcagttgctactag

SEQ ID NO: 70 - DNA, 48 bp, Artificial Sequence
  Synthetic Polynucleotide
ccaagctgaccgtcctagggtcagcccaaggctgccccctcggtcact

SEQ ID NO: 71 - DNA, 43 bp, Artificial Sequence
  Synthetic Polynucleotide
caccagctgcaccggaatgtacaccggttgcagttgctactag

SEQ ID NO: 72 - DNA, 48 bp, Artificial Sequence; identical to SEQ ID NO: 68
  Synthetic Polynucleotide

SEQ ID NO: 73 - DNA, 50 bp, Artificial Sequence
  Synthetic Polynucleotide
gactgggtcatctggatgtctgaatgtacaccggttgcagttgctactag

SEQ ID NO: 74 - DNA, 48 bp, Artificial Sequence
  Synthetic Polynucleotide
ccagggtggaggtcaaaccgtacggtggctgcaccatctgtcttcatc

SEQ ID NO: 75 - DNA, 50 bp, Artificial Sequence
  Synthetic Polynucleotide
gactccaccagccgcacctcggaatgtacaccggttgcagttgctactag

SEQ ID NO: 76 - DNA, 48 bp, Artificial Sequence; identical to SEQ ID NO: 68
  Synthetic Polynucleotide

SEQ ID NO: 77 - DNA, 53 bp, Artificial Sequence
  Synthetic Polynucleotide
ggagactgagtcatcacaatatctgaatgtacaccggttgcagttgctactag

SEQ ID NO: 78 - DNA, 50 bp, Artificial Sequence
  Synthetic Polynucleotide
gaccaagctggagatcaaaccgtacggtggctgcaccatctgtcttcatc

SEQ ID NO: 79 - protein, 125 aa, Artificial Sequence
  Synthetic
QVQLQESGPGLVKPSETLSLTCAVSGYSISDGYYWGWIRQPPGKGLEWVGSFYHGGSTYYSPSLKSRVTISIDTSRNQFSLNLNSVTGADTAVYYCARNKFLPPFTSSWYCFEYWGQGTLVTVSS

SEQ ID NO: 80 - protein, 112 aa, Artificial Sequence
  Synthetic
DIVMTQSPLSLPVTSGEPASISCRSSQSLLHSNGYNFLDWYLQKPGQSPQLLIYLGSNRASGVPDRFSGSGSGTDFILKISRVEAEDVGVYYCMQALQTPLTFGQGTKLEIK

SEQ ID NO: 81 - protein, 120 aa, Artificial Sequence
  Synthetic
EVRLVESGGGLVQPGGSLRLSCAVSGFTFSSYWMTWVRQAPGKGLEWVANIKQDGSEKNYLDSVKGRFTISRDNAKNSLYLQMNNLRVEDTALYYCARAYSGTYRAGDYWGQGTLVTVSS

SEQ ID NO: 82 - protein, 107 aa, Artificial Sequence
  Synthetic
DIQMTQSPSTLSASVGDRVTITCRASQSISAYLAWYQQKPGKAPKLLIYKASILESGVPSRFSGSGSGTEFTLSISSLQPDDFATYYCQQYNTYSQTFGQGTRVEVK

SEQ ID NO: 83 - protein, 121 aa, Artificial Sequence
  Synthetic
VQLVQSGAEVKKPGESLKISCKGSGYSFLSYWIGWVRQMPGKGLEWMGIIYPGDSDTRYSPSFEGQVTISADESLSSIFLQWSSLQASDTAMYYCARRGVGIAAAAPMDLWGQGTLVTVSS

SEQ ID NO: 84 - protein, 110 aa, Artificial Sequence
  Synthetic
QSALTQPASVSGSPGQSITISCTGTSSDIGGYKYVSWYQQHPGKAPKLMIYDVTDRPSGVSNRFSGSKSGNTASLTISGLQAEDEADYYCSSYTSSSTVIFGGGTKLTVL